(19) United States
(12) Patent Application Publication
(10) Pub. No.: US 2026/0165608 A1
(43) Pub. Date: Jun. 18, 2026

(54) METHOD FOR DETECTING AND ALLEVIATING ABNORMAL BREATHING DURING SLEEP BY USING CONTACTLESS SENSOR, AND SYSTEM THEREFOR

(71) Applicant: BRLAB INC., Seoul (KR)

(72) Inventors: Jongmin LEE, Seoul (KR); Hyunbin KWON, Seoul (KR); Kwang Suk PARK, Seoul (KR); Byung Hun CHOI, Seoul (KR)

(21) Appl. No.: 19/535,259

(22) Filed: Feb. 10, 2026

Related U.S. Application Data
(63) Continuation of application No. PCT/KR2024/012156, filed on Aug. 14, 2024.

(30) Foreign Application Priority Data
Aug. 14, 2023 (KR) 10-2023-0106503
Aug. 14, 2024 (KR) 10-2024-109390

(51) Int. Cl. A61B 5/113; A61B 5/00

(52) U.S. Cl. CPC A61B 5/113; A61B 5/486; A61B 5/6892

(57) ABSTRACT

The present invention relates to a device and method for detecting abnormal breathing during sleep by using a vibration sensor and/or a pressure sensor installed in a mattress and, when abnormal breathing is detected, providing feedback to a sleeper, so as to alleviate the abnormal breathing. The purpose of the present invention is to accurately detect abnormal breathing of a sleeping user by using a contactless sensor and provide appropriate feedback to the user, so as to improve the quality of sleep.

### CROSS-REFERENCE TO RELATED APPLICATIONS

This application is a Continuation of Application No. PCT/KR2024/012156, filed on Aug. 14, 2024, which in turn claims the benefit of Korean Patent Applications No. 10-2023-0106503, filed on Aug. 13, 2023, and No. 10-2024-0109390, filed on Aug. 13, 2024. The entire disclosures of all these applications are hereby incorporated by reference.

### TECHNICAL FIELD

The present invention relates to a device and method for detecting abnormal breathing during sleep by using a vibration sensor and/or a pressure sensor installed in a mattress and, when abnormal breathing is detected, providing feedback to a sleeper, so as to alleviate the abnormal breathing.

### BACKGROUND ART

Sleep apnea among sleep disorders is a symptom in which breathing stops or becomes shallow during sleep, which is a serious disease that may lead to various complications, such as a cardiovascular disease, stroke, diabetes, and the like. Meanwhile, the sleep apnea is generally diagnosed using polysomnography (PSG) equipment, but this method has an inconvenience of incurring inconvenience of users and generating high costs. Although portable sleep apnea diagnosis devices that can be used at home are developed recently to solve these problems, these devices still have the inconvenience of attaching sensors to the body of a user and a room for improvement from the aspect of accuracy.

The present invention relates to a method and system capable of accurately detecting abnormal breathing during sleep, more specifically, sleep apnea or snoring, while minimizing inconvenience of a user and, furthermore, providing feedback for improving these symptoms. In particular, the object of the present invention is to improve the quality of sleep by accurately detecting abnormal breathing during sleep by using contactless sensors and providing appropriate feedback to the user.

### DISCLOSURE OF INVENTION

### Technical Problem

Therefore, the present invention has been made in view of the above problems, and it is an object of the present invention to accurately detect abnormal breathing of a sleeping user by using a contactless sensor.

In addition, another object of the present invention is to provide feedback for resolving abnormal breathing according to the type of detected abnormal breathing.

Meanwhile, the technical problems of the present invention are not limited to the technical problems mentioned above, and unmentioned other technical problems can be clearly understood by those skilled in the art from the following description.

### Technical Solution

To accomplish the above objects, according to one aspect of the present invention, there is provided a system for detecting and improving abnormal breathing, the system comprising: a sensor unit provided on a predetermined layer among a plurality of layers constituting a mattress; and an operation unit for analyzing a vibration signal detected by the sensor unit and extracting biometric information including information on snoring or apnea of the user, wherein when the apnea or snoring of the user is detected from the biometric information, the operation unit generates feedback information for improving the apnea or snoring of the user.

In addition, in the system, the sensor unit may include: a vibration sensor for detecting a vibration signal; and/or a pressure sensor for detecting a pressure signal.

In addition, in the system, the vibration sensor is provided in plurality, and a first vibration sensor is provided on a predetermined layer included in the mattress, and a second vibration sensor is arranged to be stacked on the top of the mattress.

Meanwhile, a method of detecting and improving abnormal breathing according to another embodiment of the present invention is performed in a system including a sensor unit and an operation unit, and the method of detecting and improving abnormal breathing may comprise the steps of: receiving a vibration signal from a sensor unit; analyzing the received vibration signal, and extracting biometric information including information on snoring or apnea of the user; and generating feedback information for improving the apnea or snoring of the user when the apnea or snoring of the user is detected from the biometric information.

### Advantageous Effects

The present invention has an effect of minimizing inconvenience of a user when grasping the sleeping state of a user by utilizing contactless sensors.

In addition, the present invention has an effect of detecting abnormal breathing at a high accuracy although contactless sensors are used.

In addition, the present invention has an effect of preventing the problem of privacy infringement of a user as sound recording of a sleeping user is not required.

In addition, the present invention may effectively contribute to improving the sleep quality of a user as feedback can be provided in a customized manner according to the type of detected abnormal breathing.

Meanwhile, the effects of the present invention are not limited to those mentioned above, and unmentioned other technical effects will be clearly understood by those skilled in the art from the following description.

### BRIEF DESCRIPTION OF THE DRAWINGS

FIG. 1 is a view for explaining the overview of a system for detecting and improving abnormal breathing according to the present invention.

FIG. 2 is a flowchart sequentially illustrating the steps of a method of detecting and improving abnormal breathing according to an embodiment of the present invention.

FIG. 3 is a view for explaining the location of vibration sensors in a mattress according to an embodiment.

FIG. 4 is a view for explaining the position of vibration sensors provided in a mattress according to another embodiment.

### BEST MODE FOR CARRYING OUT THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

A system for detecting and improving abnormal breathing according to the present invention (hereinafter referred to as a “system” for convenience) may include detection means existing in a mattress to detect information on the state of a user, for example, a sensor for detecting pressure (pressure sensor) or a sensor for detecting vibration (vibration sensor). At this point, the sensors may be included in an upper part of the mattress (the part in contact with the top surface) and/or any one layer among a plurality of layers constituting the mattress. Furthermore, the system may include an operation unit required to control the components. In addition, a communication unit for receiving control commands from the outside or transmitting monitoring information to the outside may be further provided as needed. In addition, the present invention may be based on the premise that a vibration generating means is additionally provided in the mattress.

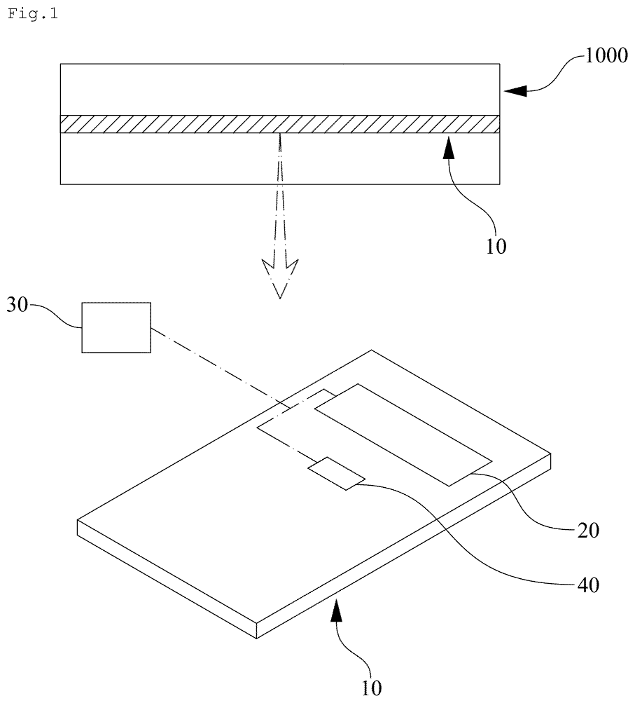

FIG. 1 is view for explaining the basic configuration of the system 100 according to the present invention. The upper part of FIG. 1 shows a view in which a predetermined layer 10 is included in a mattress 1000, and the lower part of FIG. 1 shows a view in which a sensor unit 20 is provided on the layer 10 and the sensor unit 20 is connected to an operation unit 30. In addition, the system 100 according to the present invention may further include a feedback provision unit 40 for providing feedback to the user. In the detailed description, it will be described on the premise that the sensor unit 20, the operation unit 30, and the feedback provision unit 40 constitute one system 100.

The layer 10 may be understood as a member having an accommodation space in which the components that will be described below can be arranged, and the material or shape of the layer 10 is not limited as long as the accommodation space is provided. The layer 10 may be any one among a plurality of surfaces constituting the mattress 1000, and in some cases, the layer 10 may be the mattress 1000 itself. In addition, the layer 10 may be a mat that can be placed on the mattress, and further, it may be a member made of wood or metal, rather than a surface of a fiber material. As described, the material or shape of the layer 10 is not limited as long as a predetermined accommodation space is provided. In the detailed description, in order to help understanding of the invention, it will be described on the premise that the layer 10 is one layer constituting the mattress 1000, and it is to be understood that a plurality of layers may be included in the mattress 1000 as a stacked structure.

The sensor unit 20 is provided in the mattress 1000, and it is a component that detects and acquires biometric information from the user. Specifically, the sensor unit 20 may broadly include a vibration sensor for detecting vibration and a pressure sensor for detecting pressure.

The vibration sensor may be implemented using at least one among Polyvinylidene fluoride (PVDF), an EMFi sensor, a piezoelectric sensor, an air cell sensor, a load cell sensor, an acceleration sensor, a strain gauge, and an FSR sensor. Preferably, the vibration sensor may be implemented using PVDF, and the PVDF is a piezoelectric material that generates electricity when a mechanical/physical stimulus is applied, and may detect vibration signals from a user lying on the mattress by utilizing this property. The vibration sensor may detect vibration to obtain biometric information of the user, particularly at least one among ballistic cardiogram, respiratory state, and movement state.

The pressure sensor may be implemented using an FSR, an air cell sensor, or the like, and preferably implemented using a Force Sensing Resistor (FSR) array. The FSR is a component that converts a pressure value into a resistance value, and when a plurality of FSRs are provided in the form of an array, the posture of the user can be estimated using this property with reference to the magnitude of the resistance values detected while the user is lying on the mattress.

The operation unit 30 may detect abnormal breathing of a user from the vibration signal and/or pressure signal acquired by the sensor unit 20, and may also generate feedback information for resolving the detected abnormal breathing problem. At this point, the feedback information may be a control command related to the type of a stimulus to be applied to the user to resolve the detected abnormal breathing problem. That is, the feedback information may be a control command needed to provide a feedback stimulus to a user showing abnormal breathing through the feedback provision unit 40 mentioned above. On the other hand, the feedback information may not be a control command for controlling a specific feedback provision unit 40, rather it may be a collection of information including an evaluation of the overall sleeping state of the user, a result of whether abnormal breathing is detected, information on the type of detected abnormal breathing, and/or matters needed to improve abnormal breathing. It may be understood that the feedback information at this point is provided in a form that can be output to the terminal (smartphone, computer, etc.) of the user so that the user may confirm his/her sleeping state, whether abnormal breathing is detected, or the like.

Meanwhile, the operation unit 30 may also be understood as a central processing unit, in which a predetermined data analysis and calculation process is performed. The central processing unit may also be referred to as a controller, a microcontroller, a microprocessor, a microcomputer, or the like. In addition, the central processing unit may be implemented by hardware, firmware, software, or a combination thereof. In the case of implementing using hardware, the central processing unit may be configured as an application specific integrated circuit (ASIC), a digital signal processor (DSP), a digital signal processing device (DSPD), a programmable logic device (PLD), a field programmable gate array (FPGA), or the like. In the case of implementing using firmware or software, the firmware or software may be configured to include modules, procedures, functions, or the like that perform the functions or operations described above. In addition, the operation unit 30 may also include a memory, and the memory may be implemented as a Read Only Memory (ROM), a Random Access Memory (RAM), an Erasable Programmable Read Only Memory (EPROM), an Electrically Erasable Programmable Read-Only Memory (EEPROM), a flash memory, a Static RAM (SRAM), a Hard Disk Drive (HDD), a Solid State Drive (SSD), or the like.

The operation unit 30 may extract meaningful signals from the signals obtained by the detection units through filtering, and may analyze the current sleeping state of the user or detect abnormal breathing on the basis of at least one signal among the extracted signals or a combination of a plurality of meaningful signals. In addition, the operation unit 30 may also generate feedback information matching the type of detected abnormal breathing according to the type of abnormal breathing.

The feedback provision unit 40 may be understood as a configuration that generates a predetermined stimulus to improve the sleeping state of the user according to whether abnormal breathing is detected. The feedback provision unit 40 may be implemented in various ways according to the type of stimulus that may affect the user. For example, it may be implemented as an actuator that may generate vibration, a speaker that may generate sounds, a heater that may generate heat, a cooler that may generate cold air, a fan that may generate wind, a lighting device that may generate light, an actuator that may generate a curvature of the mattress surface to induce the sleeping posture of the user to another posture, or a component that may stimulate at least one of human senses. The feedback provision unit 40 does not necessarily present only in the mattress 1000, and may exist at a place spaced apart from the mattress 1000 as much as a predetermined distance.

The schematic configuration of the system 100 according to the present invention has been described with reference to FIG. 1.

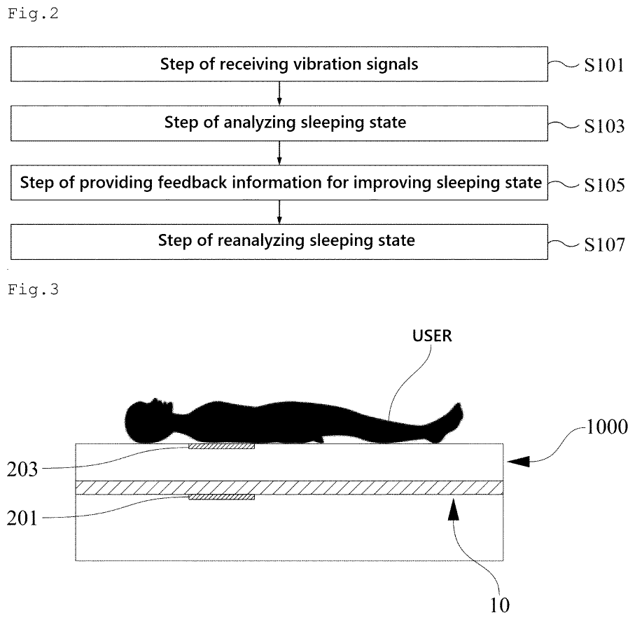

FIG. 2 is a view showing an embodiment in which vibration sensors are provided in the mattress 1000. Specifically, the system 100 according to the present invention may be characterized in that vibration sensors 201 and 203 are arranged on the top of the mattress 1000 (the part in contact with the uppermost surface of the mattress) and on a predetermined layer 10 constituting the mattress. In the drawing, it can be confirmed that a first vibration sensor 201 is provided to be in contact with the layer 10, and the second vibration sensor 203 is provided on the top of the mattress.

When the vibration sensor is arranged only on the top of the mattress, the vibration sensor is tightly attached to the human body, so that vibrations generated by heartbeats and changes in the volume generated by breathing may be detected by the sensor further better. In addition, vibrations generated by snoring may also be transmitted to the vibration sensor more accurately, and therefore, the accuracy of detecting biometric signal/biometric information can be enhanced. However, when the vibration sensor is arranged on the top of the mattress, even a small movement of the sleeper may be detected by the vibration sensor, and thus there may be a negative effect of increasing noise to some extent from the aspect of extracting the biometric signal/biometric information. In addition, when sleep apnea is generated from a sleeper, the sleeper stops breathing and then suddenly gasps as oxygen demand increases, and thus it is worried that a situation of detecting a signal larger than normal breathing is generated by the vibration sensor, and the sleep apnea situation is considered as a noise.

Meanwhile, when the vibration sensor is arranged only in the layer 10 in the mattress, it can be implemented so that another layer, e.g., a mattress foam, may function as a physical filter. That is, the vibration signal may be attenuated as it passes through the path of [sleeper-mattress foam-vibration sensor], and this may be understood as a view of utilizing the mattress foam as a type of filter. For reference, the mattress foam refers to, for example, only one of several layers included in the mattress, and of course, another layer, other than the mattress foam, may function as a physical filter. The advantage at this point is that when an apnea patient suddenly gasps after stopping breathing, the signal is measured at a relatively small magnitude and is not considered as a noise. The disadvantage is that the magnitude of the biometric signal is also attenuated, and thus accuracy of biometric information measurement can be lowered. In addition, there may be a problem in that as the mattress foam absorbs vibrations (high-frequency signals) generated by snoring, the vibration sensor may not detect a snoring signal. Accordingly, the present invention is to utilize the advantages and compensate for the disadvantages by arranging a plurality of vibration sensors on the top and in the middle of a mattress.

Assuming that the vibration sensors are arranged both on the top of the mattress and the layer in the middle of the mattress, for example, when large vibration signals are detected from the vibration sensor on the surface (top) of the mattress and also from the vibration sensor provided on the layer in the middle, the system may determine that the sleeper has moved. In addition, the system may be implemented, when a large vibration signal is detected from the vibration sensor on the surface of the mattress, but a large vibration signal is not detected from the vibration sensor provided on the layer in the middle, to determine the situation as snoring or apnea. In this way, the system according to the present invention may relatively accurately detect abnormal breathing of a user.

On the other hand, it is preferable to arrange the first vibration sensor 201 and the second vibration sensor 203 so that at least a portion of the areas overlap when the mattress 1000 is seen down from a flat surface. When the first vibration sensor 201 and the second vibration sensor 203 are arranged to be spaced far apart from each other, for example, when the first vibration sensor 201 is arranged to hang over the user's shoulder while the second vibration sensor 203 is arranged to be in contact with the user's calf, signal analysis and abnormal breathing detection based on the collected vibration signals may not be possible as the vibration signals are detected from different areas of the user's body. Therefore, in order to match the temporal and spatial conditions of the vibration signals acquired from the mattress 1000 as much as possible, it is preferable to arrange the first vibration sensor 201 and the second vibration sensor 203 so that at least a portion of the areas overlap when the mattress is seen down from a flat surface.

FIG. 3 is a flowchart sequentially illustrating a method of detecting and improving abnormal breathing according to an embodiment of the present invention. Referring to the drawing, the method of detecting and improving abnormal breathing may first include a step of receiving a vibration signal from a vibration sensor(s) (S101), and a step of analyzing the sleeping state of a user by analyzing the received vibration signal (S103), more precisely, a step of detecting abnormal breathing.

At the step of detecting abnormal breathing, typically, snoring and/or apnea phenomena are detected first, and whether abnormal breathing is detected may be determined accordingly. Snoring is generating a noise when air passes through a narrow airway, and it is known to generate vibration of the human body. The vibration may be measured by vibration sensor(s) and separated by performing signal processing at a frequency higher than that of biometric signals (e.g., ballistic cardiogram, respiration). That is, snoring can be detected in real time by applying a relatively high-pass filter to the signals acquired by the vibration sensors 201 and 203 and then quantifying the magnitude and periodicity of the signals. Apnea refers to a state in which breathing stops during sleep (central apnea) or breathing is impossible or difficult as the airway is completely or partially obstructed (obstructive apnea). It is known that when apnea occurs, there is a difference in the change of volume of the thoracic cavity compared to a normal breathing situation. Referring to this, it can be seen that when filtering is applied to the signals measured by the vibration sensors 201 and 203 with the respiratory band, there is a difference in the magnitude and periodicity of the signal in the cases of normal breathing and apnea, and also a phenomenon of rapid breathing appears after apnea, and since this may also be measured by the vibration sensor (the magnitude and frequency characteristics of the signal are different from those of normal breathing), apnea of a sleeper can be detected. That is, normal breathing and apnea can be evaluated in real time by quantifying the characteristics such as the magnitude, frequency characteristics, and periodicity of the signals processed in the respiratory band.

Meanwhile, although it is described in FIG. 3 that only vibration signals are received at step S101, pressure signals acquired from a pressure sensor (not shown) may also be utilized as needed in the process of detecting abnormal breathing of the user. Although the pressure sensor is provided to detect changes in the movement and posture of the sleeper, it is also possible to extract a signal that can identify the breathing state of the user from the pressure signal acquired by the pressure sensor, and in the present invention, the vibration sensor and the pressure sensor may be implemented to be used to complement each other to detect snoring/apnea.

The system 100 may be implemented to generate feedback information for improving the sleeping state and provide feedback to the user through this when the sleeping state is analyzed at step S103 (S105). That is, it can be implemented to provide a stimulus according to the type of snoring or apnea when snoring or apnea is detected in real time as a result of analyzing the sleeping state of the user.

The sleep apnea is broadly divided into central apnea and obstructive apnea. The central apnea refers to a case where a command to breathe does not reach, and thus there is no airflow measured from the oral-nasal cavity and no respiratory effort measured from the chest, and the obstructive apnea refers to a case where the airway is obstructed, and although there is no airflow measured from the oral-nasal cavity, there is a respiratory effort measured from the chest. In addition, the apnea may also be further classified into apnea and hypopnea, and they are evaluated based on the percentage of decrease in the magnitude of the current respiratory signal compared to the previous normal respiratory signal.

The system 100 according to the present invention may detect abnormal breathing in the manner described above, and it may be implemented to provide, when abnormal breathing (apnea) is detected, feedback for improvement that varies according to the classification of the apnea. For example, when the apnea of the sleeper currently evaluated in real time is obstructive apnea, feedback may be provided to turn the head of the sleeper placed on the pillow to secure the airway. The types of feedback may include images and text displayed on the screen, sounds output through a speaker, vibration stimulus output by a vibration means provided on the mattress, operation of an actuator for changing the curvature of the mattress surface, and the like. Meanwhile, when the current state of the sleeper is obstructive apnea and the sleeper is in the supine position, a vibration stimulus that the sleeper does not feel (the criterion of this vibration stimulus is predetermined according to learning after the biometric signals of the user are acquired during sleep) may be generated to induce the sleeper to take a lateral position and secure the airway. Meanwhile, the system may be implemented to provide a vibration stimulus to the sleeper so that the user may feel and generate a breathing command from the brain when the central apnea occurs.

In addition, it is known that activities of the sympathetic nervous system increase during sleep in the case of a sleep apnea patient, and the system according to the present invention may be implemented to reduce the frequency of snoring/apnea by increasing the activities of the parasympathetic nervous system of the sleeper by using a vibration stimulation method synchronized with heart rate.

After step S105, a step of reanalyzing the sleeping state of the user, i.e., a step of grasping again whether the abnormal breathing of the user is improved or the overall sleeping state is improved after the feedback is provided, may be executed (S107).

This step may also be implemented to adjust in real time the feedback provided to the user. For example, characteristics such as the intensity and frequency of the feedback provided to the user may be adjusted by reflecting the condition of the user in real time, and it may be adjusted to provide feedback suitable for the condition of the user in a method of decreasing the intensity of the stimulus next time when the sleeper wakes up after a vibration stimulus is applied, and gradually increasing the intensity after a predetermined period of time when there is no response although a vibration stimulus is applied.

In addition, the system may be implemented to store, at step S107, information such as whether the abnormal condition is improved, the number of times of providing feedback for improvement, and intensity of the provided stimulus when feedback is provided with respect to the abnormal breathing (snoring/apnea) of the user. This information may be utilized to classify the abnormal breathing in detail, to match abnormal breathing with feedback for improvement, to conduct a specific diagnose, and to suggest activities during the daytime. For example, as the user snores while being in a supine position, a vibration notification is provided to induce a change in the posture, and when the user changes the posture and the snoring stops, it can be seen that the snoring phenomenon can be improved with the feedback for change in the posture. As another example, as the user snores while being in a supine position, a vibration notification is provided to induce a change in the posture, and when the snoring is maintained although the posture is changed, it can be seen that the snoring cannot be improved with the feedback. This information may be used as a reference for suggesting another feedback or generate guidance for weight loss or surgery as the airway is still blocked.

The method of detecting and improving abnormal breathing according to the present invention has been described above with reference to FIG. 3.

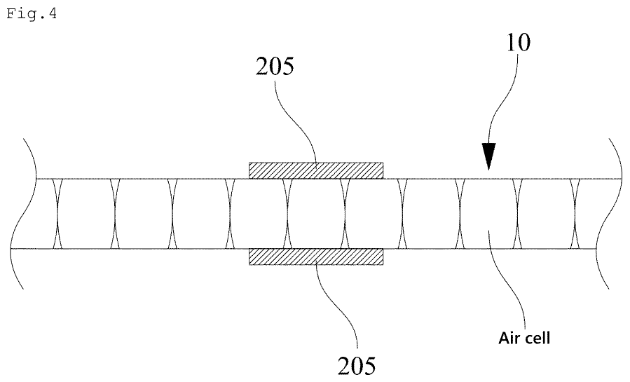

Finally, FIG. 4 is a view showing a system configuration according to another embodiment of the present invention. The system 100 according to the present invention may be implemented in combination with members having additional functions. For example, the vibration sensor utilized in the present invention may have a thin surface shape in the structure, and thus may be used together with various members on the mattress. As a more specific example, air cells may be provided on the mattress to adjust firmness of the mattress that the user feels, and although such air cells are provided, biometric signals or biometric information may be collected by arranging a vibration sensor 205 on the contact surface of the layer including the air cells. Although the drawing shows an embodiment in which the vibration sensor 205 is provided on the top and bottom surfaces of the layer including the air cells, it should be understood that there may also be an embodiment in which the vibration sensor is provided on only one side of the layer.

Furthermore, hardness of the air cells may be adjusted by injecting or exhausting air according to the body pressure, posture, or request of the user. The system 100 according to the present invention may be implemented to prevent incorrect detection of biometric information (heart rate, respiration rate, etc.) by additionally collecting state information of the air cells and controlling the vibration sensor 205 to stop measuring biometric signals when the state of the air cells is changing for adjustment of hardness, i.e., while being in operation.

In addition, the system 100 according to the present invention may be implemented to measure the movement, breathing, or heartbeat of the user through a change in the pressure of the air cells. When a minute vibration due to the heartbeat, change in the volume due to breathing, or movement of the sleeper is generated, a force (pressure) is applied to the air cells, and a change in the air pressure inside the air cells may occur, and the system may be implemented to detect the change in the air pressure using a hydraulic sensor, and as the gain of the biometric signal measurement unit is adaptively adjusted according to the level of air pressure in the air cells, the biometric signal or biometric information can be acquired more accurately from the sleeper.

A method and system for detecting and improving abnormal breathing according to the present invention have been described above. Meanwhile, the present invention is not limited to the specific embodiments and application examples described above, and it goes without saying that various modified embodiments can be made by those skilled in the art, and the modified embodiments should not be understood as being distinguished from the technical spirit or prospect of the present invention.

In particular, the components that implement the technical features of the present invention included in the block diagrams and flowcharts illustrated in the drawings attached to this specification mean logical boundaries between the components. However, according to an embodiment of software or hardware, the illustrated components and their functions are implemented in the form of standalone software modules, monolithic software structures, codes, services, and combinations thereof, and as they are stored in a medium that can be executed in a computer having a processor capable of executing the stored program codes, instructions, and the like to implement the functions, all of these embodiments should also be construed as falling within the scope of the present invention.

Accordingly, although the attached drawings and description thereof illustrate technical features of the present invention, they should not be simply inferred unless specific arrangements of software for implementing such technical features are explicitly mentioned. That is, various embodiments as described above may exist, and since such embodiments can be modified partly while having the same technical features as the present invention, they should also be construed as falling within the scope of the present invention.

In addition, although the flowcharts illustrate operations in the drawings in a particular order, this is merely intended to achieve the most desirable results and should not be construed as requiring execution of the operations in the illustrated particular or sequential order or requiring execution of all the illustrated operations. In specific cases, multitasking and parallel processing may be advantageous. Furthermore, separation of various system components of the embodiments described above should not be construed as requiring such separation in all embodiments, and it should be understood that it is general for the program components and systems described above to be integrated together in a single software product or packaged in a plurality of software products.

### DESCRIPTION OF SYMBOLS

1000: Mattress
10: Layers
20: Sensor unit
201: First vibration sensor
203: Second vibration sensor
30: Operation unit
40: Feedback provision unit

1. A system for detecting and improving abnormal breathing for detecting and improving abnormal breathing of a sleeping user, the system comprising:
a sensor unit provided on a predetermined layer among a plurality of layers constituting a mattress; and
an operation unit for analyzing a vibration signal detected by the sensor unit and extracting biometric information including information on snoring or apnea of the user, wherein
when the apnea or snoring of the user is detected from the biometric information, the operation unit generates feedback information for improving the apnea or snoring of the user.

2. The system according to claim 1, wherein the sensor unit includes:
a vibration sensor for detecting a vibration signal; and/or
a pressure sensor for detecting a pressure signal.

3. The system according to claim 2, wherein the vibration sensor is formed in plurality, wherein a first vibration sensor is provided on a predetermined layer in the mattress, and a second vibration sensor is provided on the top of the mattress.

4. A method of detecting and improving abnormal breathing of a user, which is performed in a system including a sensor unit and an operation unit, the method comprising the steps of:
receiving a vibration signal from a sensor unit;
analyzing the received vibration signal, and extracting biometric information including information on snoring or apnea of the user; and
generating feedback information for improving the apnea or snoring of the user when the apnea or snoring of the user is detected from the biometric information.

5. The method according to claim 4, wherein the sensor unit includes:
a vibration sensor for detecting a vibration signal; and/or
a pressure sensor for detecting a pressure signal.
